(19) United States
(12) Patent Application Publication
(10) Pub. No.: US 2016/0235824 A1
(43) Pub. Date: Aug. 18, 2016

(54) Compositions and Methods for Increasing the Half-Life of Factor XA

(71) Applicant: THE CHILDREN'S HOSPITAL OF PHILADELPHIA, Philadelphia, PA (US)

(72) Inventors: Rodney M Camire, Sicklerville, NJ (US); Nabil K. Thalji, Philadelphia, PA (US)

(73) Assignee: The Children's Hospital Philadelphia, Philadelphia, PA (US)

(21) Appl. No.: 15/031,077

(22) PCT Filed: Nov. 3, 2014
(86) PCT No.: PCT/US2014/063676

§ 371 Date: Apr. 21, 2016

Related U.S. Application Data
(60) Provisional application No. 61/898,884, filed on Nov. 1, 2013.
(60) Provisional application No. 61/918,341, filed on Dec. 19, 2013.

(51) Int. Cl. A61K 38/48; A61K 31/4545; A61K 31/5377

(52) U.S. Cl. CPC A61K 38/4846; A61K 31/5377; A61K 31/4545

(57) ABSTRACT

Compositions and methods for the modulation of hemostasis are disclosed.

This application claims priority under 35 U.S.C. §119(e) to U.S. Provisional Patent Application No. 61/898,884, filed Nov. 1, 2013, and U.S. Provisional Patent Application No. 61/918,341, filed Dec. 19, 2013. The foregoing application is incorporated by reference herein.

This invention was made with government support under Grant Numbers P01 HL-74124 awarded by the National Institutes of Health. The government has certain rights in the invention.

### FIELD OF THE INVENTION

The present invention relates to the fields of medicine and hematology. More specifically, the invention provides compositions and methods for increasing the half-life of Factor Xa and variants thereof. Methods of using the same to modulate the coagulation cascade and treat hemostasis related disorders in a subject are also provided.

### BACKGROUND OF THE INVENTION

Several publications and patent documents are cited throughout the specification in order to describe the state of the art to which this invention pertains. Each of these citations is incorporated herein by reference as though set forth in full.

Hemostasis is an essential component of cardiovascular homeostasis that prevents bleeding at the site of vascular injury while maintaining vascular patency. The process is tightly controlled to prevent thrombosis (pathological, excessive clotting) or bleeding. Hemostasis consists of two components, primary hemostasis that results in aggregation of activated platelets to form an initial platelet plug, and secondary hemostasis where a stable clot consisting primarily of cross-linked fibrin polymers is deposited at the site of injury. Secondary hemostasis is achieved by a cascade of homologous serine proteases and their cofactors that result in formation of the procoagulant serine protease, thrombin. Thrombin generated by the clotting cascade converts soluble fibrinogen into insoluble fibrin that polymerizes to form a clot. The hemostatic system also consists of circulating coagulation inhibitors to prevent indiscriminate coagulation that would compromise vessel patency. Furthermore, coagulation serine proteases and cofactors are synthesized in the liver as inactive zymogens and procofactors, respectively. Zymogens and procofactors undergo site-specific proteolytic cleavage that yields an active protease or cofactor.

Pharmacological anticoagulation is the mainstay of treatment for patients with prothrombotic conditions, including those with atrial fibrillation, history of pulmonary embolism or deep venous thrombosis, and patients with prosthetic heart valves. For over 50 years, the only oral anticoagulant available was warfarin, an inhibitor of the vitamin K epoxide reductase (VKOR) that recycles oxidized vitamin K. Vitamin K is a critical cofactor in the post-translational γ-carboxylation of several coagulation serine proteases, and thus warfarin is a potent anticoagulant. Unfortunately, the use of warfarin has many drawbacks, including its complex and unpredictable pharmacokinetics that necessitate frequent monitoring of coagulation parameters and dose adjustment. However, in the event of emergency bleeding or the need for urgent or emergent surgery, antidotes exist that allow rapid and complete reversal.

Recently, oral anticoagulants directly inhibiting thrombin and FXa have been developed that have much more predictable pharmacokinetics, simpler dosing schemes, and fast onset and offset compared to warfarin. Oral direct FXa inhibitors, including rivaroxaban and apixaban, are important new drugs that are noncompetitive inhibitors of FXa with respect to prothrombin. They bind in the substrate binding cleft and inhibit FXa competitively with respect to small peptidyl substrates that also bind in the substrate binding cleft. Both apixaban and rivaroxaban inhibit the enzyme with high picomolar/low nanomolar Kᵢ values, and are heavily protein-bound in plasma. While oral FXa inhibitors possess advantages over warfarin, there is currently no fully efficacious reversal agent for oral direct FXa inhibitors. Inasmuch as a reversal agent is desired to reverse uncontrolled bleeding from the use of the direct FXa inhibitor, this represents an unmet clinical need.

With regard to pro-coagulation, the response to damage needs to be focused and commensurate with the extent of injury. As stated hereinabove, coagulation proceeds through a series of proteolytic reactions involving enzymes that become activated, culminating in the generation of the final enzyme thrombin which activates platelets and cleaves a structural protein (fibrinogen) to generate a fibrin, providing a meshwork which physically prevents blood from leaving the vessel. Deficiency of proteins that lead to the formation of thrombin can cause bleeding complications. One of the most common types of bleeding disorders is hemophilia A and B. Hemophilia A is characterized by a deficiency in coagulation factor VIII and hemophilia B is characterized by factor IX deficiency. Current therapy for hemophilia is carried out by replacement of the defective or missing coagulation factors. Unfortunately, some patients (˜3-20%) develop high-titer, inhibitory antibodies to the infused factor VIII or factor IX. Development of inhibitors against the administrated proteins represents a severe problem in the management of hemophilia. In these so-called inhibitor patients alternative strategies have been developed which bypass the intrinsic pathway such as activated prothrombin complex concentrates (aPCCs) and recombinant FVIIa (NovoSeven®). These products work by accelerating FXa formation and ultimately thrombin generation thereby providing adequate hemostasis. Because of a whole host of issues including short half-life, effective dose range, cost and potential for thrombotic complications other approaches should be explored. An alternative approach could be to infuse FXa directly. However, it has a very short half-life in plasma. Accordingly, there is still an urgent unmet clinical need.

### SUMMARY OF THE INVENTION

In accordance with the instant invention, methods of modulating hemostasis in a subject are provided. In a particular embodiment, methods for inhibiting, treating, and/or preventing a hemostasis related disorder in a subject are provided. In a particular embodiment, the method comprises the administration of a therapeutically effective amount of a Factor Xa or a variant thereof and a direct FXa inhibitor. The Factor Xa or a variant thereof and the direct FXa inhibitor can be administered simultaneously and/or sequentially. In a particular embodiment, the direct FXa inhibitor is selected from the group consisting of apixaban, betrixaban, darexaban, edoxaban, otamixaban, and rivaroxaban. In a particular embodiment, the Factor Xa variant comprises a substitution at position 16 or 17, particularly the variant comprises a Leu at position 16 in chymotrypsin numbering system.

In accordance with another aspect of the instant invention, methods are provided for reducing the anticoagulation effect of a direct FXa inhibitor in blood. In a particular embodiment, the method comprises contacting the blood with an effective amount of Factor Xa or a variant thereof. The method may be performed in vitro or in vivo. In a particular embodiment, the direct FXa inhibitor is selected from the group consisting of apixaban, betrixaban, darexaban, edoxaban, otamixaban, and rivaroxaban. In a particular embodiment, the Factor Xa variant comprises a substitution at position 16 or 17, particularly the variant comprises a Leu at position 16 in chymotrypsin numbering system.

In accordance with another aspect of the instant invention, compositions for the modulation of blood coagulation are provided. In a particular embodiment, the composition comprises at least one Factor Xa or variant thereof and at least one direct FXa inhibitor. The composition may further comprising at least one pharmaceutically acceptable carrier.

### BRIEF DESCRIPTION OF THE DRAWINGS

FIGS. 1A and 1B provide graphs of thrombin generation assays (TGA) performed using rivaroxaban and FXa^{I[16]L}. FIG. 1A shows the titration of rivaroxaban into platelet poor plasma (PPP), demonstrating sensitivity of the assay to inhibitor levels. FIG. 1B shows the titration of FXa^{I[16]L} into PPP spiked with 500 nM rivaroxaban. Data is quantified as peak thrombin generation expressed as a percentage of normal peak thrombin generation. 1 nM of the variant is sufficient to restore thrombin generation in this assay.

FIG. 2 provides a graph of rotational thromboelastograms performed using freshly collected, citrated human blood treated with 25 μg/mL corn trypsin inhibitor (to minimize variation in samples). 250 nM apixaban was added to blood and concentrations of FXa^{I[16]L} (0.3 nM and 3 nM) were added to reverse the effects. Phosphate buffered saline (PBS) was added to one sample instead of apixaban as a positive control. Apixaban prolonged the clot time substantially and 0.3 nM variant was sufficient to restore normal hemostasis.

FIGS. 3A and 3B show the effect of FXa pre-incubation in plasma containing rivaroxaban. WT FXa (right bars) or the Ile[16]Leu variant (left bars) were added to PPP in the presence (FIG. 3A) or the absence (FIG. 3B) of 500 nM rivaroxaban. After the noted incubation time, TGA reactions were initiated by the addition of the tissue factor/phospholipids and the TGA substrate. In FIG. 3B, 500 nM rivaroxaban was added along with the other TGA reagents prior to starting the reaction. Incubation time is plotted against peak thrombin generation as a percentage of that of PBS-spiked PPP. The sample labeled “500 nM riv” represents PPP treated with only 500 nM rivaroxaban and PBS instead of FXa.

FIG. 4A provides an amino acid sequence of human Pre-Pro-Factor X (SEQ ID NO: 2). The underlined and bolded residues are positions 16, 17, 18, 19, and 194 in chymotrypsin numbering. FIG. 4B provides an amino acid sequence of the light chain (SEQ ID NO: 3) and heavy chain (SEQ ID NO: 4) of Factor X. FIG. 4C provides an amino acid sequence of the light chain (SEQ ID NO: 3) and heavy chain (SEQ ID NO: 5) of activated Factor X (FXa). FIG. 4D provides a nucleic acid sequence (SEQ ID NO: 6) which encodes human FX preproprotein.

FIG. 5 provides a graph of FXa-antithrombin III (FXa-ATIII) complex formation as a function of incubation time. 25 nM WT FXa was added to FX deficient plasma containing no rivaroxaban, 100 nM rivaroxaban, or 1 μM rivaroxaban. Samples were incubated for the indicated time and then all residual FXa was quenched by addition of 50 μM biotinylated glutamyl-glycinyl-arginyl-chloromethylketone (BEGRCK), which irreversibly modifies the active site of FXa and prevents further reaction with antithrombin III. FXa-ATIII levels were then measured with an enzyme-linked immunosorbent assay (ELISA) using an anti-FX capture antibody and an HRP-anti-ATIII detection antibody.

### DETAILED DESCRIPTION OF THE INVENTION

Coagulation serine proteases are critical components of the hemostatic process that leads to formation of a stable blood clot upon vascular injury. This process must be tightly controlled to prevent excessive coagulation (thrombosis) or insufficient coagulation (bleeding). Major elements of this control include synthesis of the proteases as inactive zymogens that can be activated locally by proteolytic cleavage following vascular damage, and the presence of plasma protease inhibitors that rapidly inactivate free activated proteases. Insufficient regulation of coagulation leads to prothrombotic conditions including atrial fibrillation, pulmonary embolism, and deep venous thrombosis. Pharmacological anticoagulation is the mainstay of treatment for these patients. Recently, new oral anticoagulants that directly inhibit coagulation factor Xa (FXa), the penultimate protease in the clotting cascade, have emerged that promise to simplify treatment regimens. While these direct FXa inhibitors have been shown to be highly efficacious with at least a comparable safety profile to warfarin, the most widely used oral anticoagulant, the lack of specific countermeasures to their effects in the event of major bleeding or emergency surgery is a major concern for physicians.

FXa is the critical serine protease that, along with its cofactor FVa, proteolytically activates prothrombin, to generate thrombin. The FX zymogen is activated by the intrinsic or extrinsic pathway of coagulation by specific cleavage and removal of an activation peptide. Removal of the activation peptide leads to a conformational change, termed the zymogen-to-protease transition, that yields the active protease. Variants of FXa with an impaired ability to undergo this critical conformational change have been developed. These variants have been shown to have therapeutic potential as procoagulants in the setting of hemophilia or intracranial hemorrhage based on their unique biochemical properties. These zymogen-like FXa variants may also serve as effective countermeasures to direct FXa inhibitors. Herein, it is demonstrated how FXa and variants thereof behave in terms of catalytic function and half-life in the presence of direct FXa inhibitors.

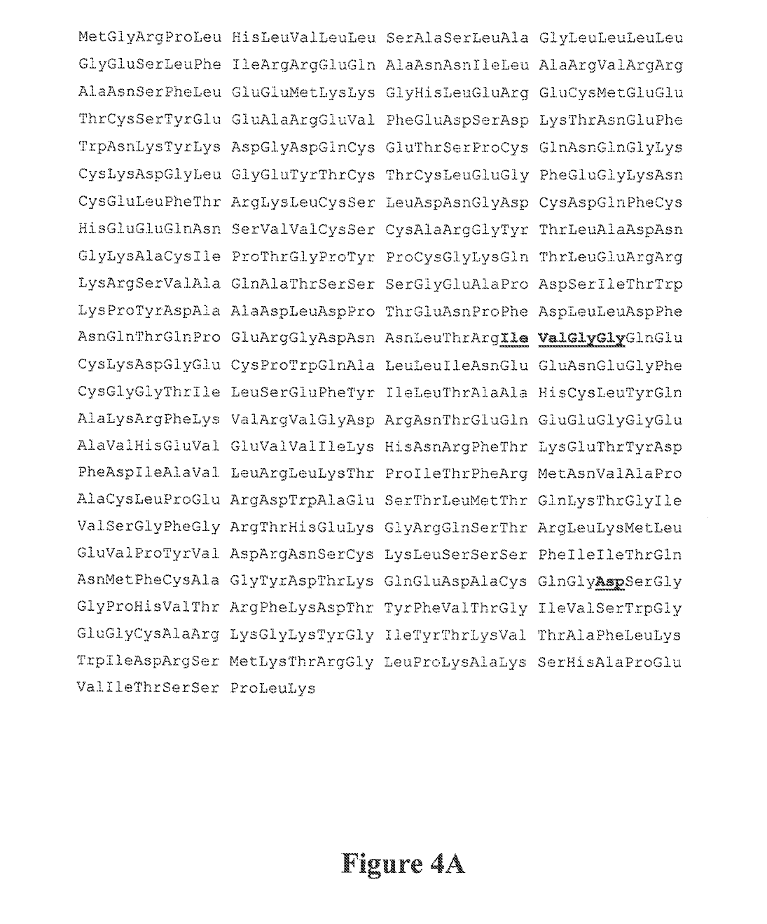

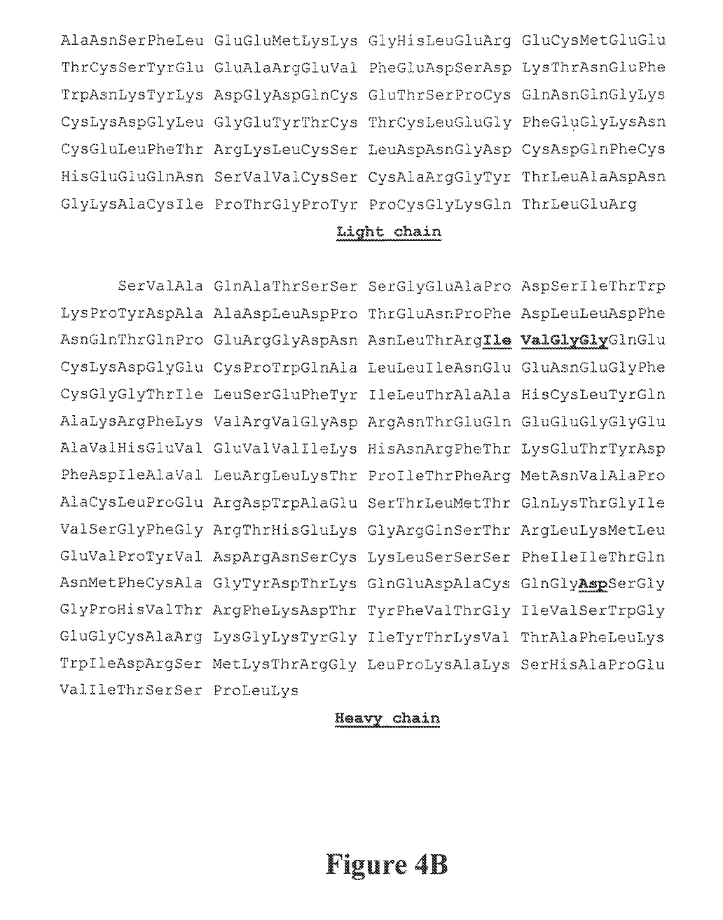

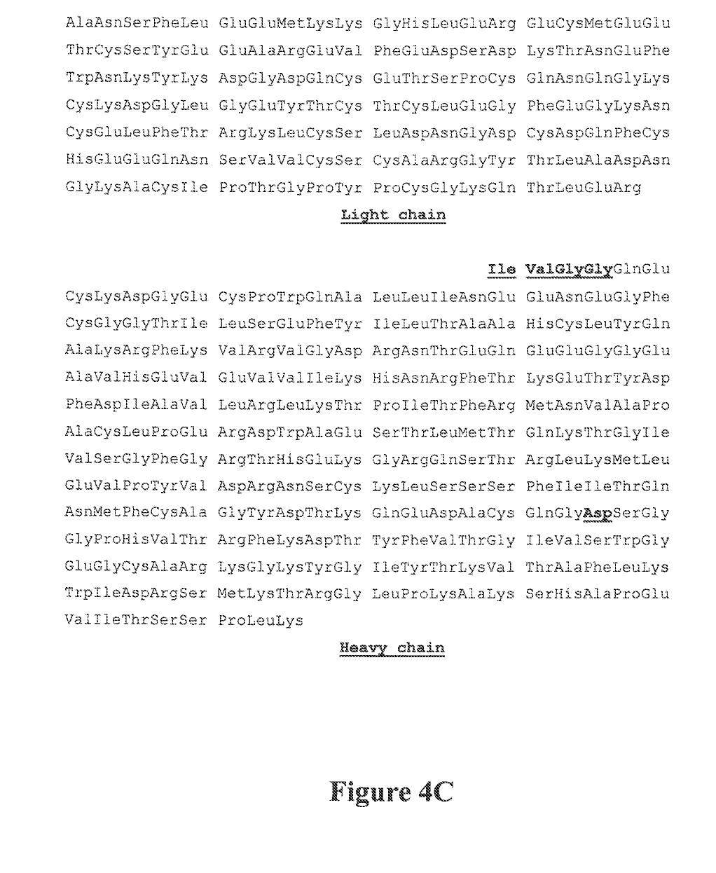

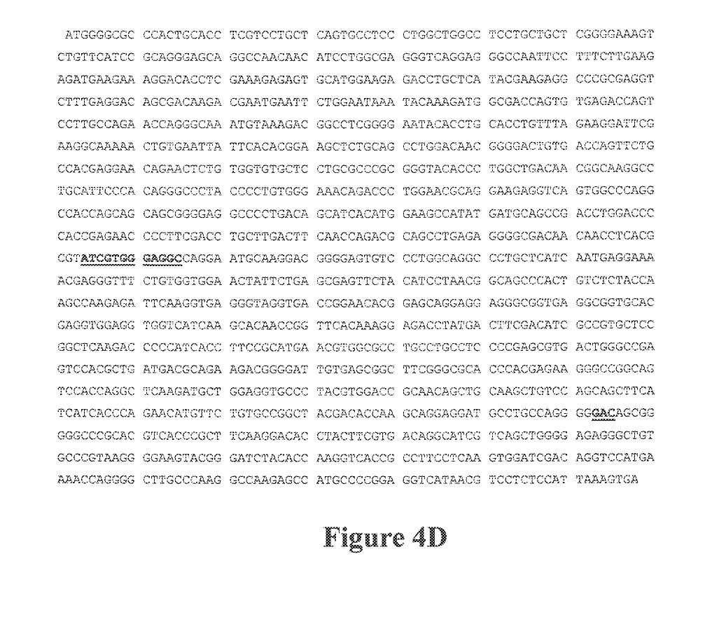

Factor Xa (FXa) is a key serine protease in the clotting cascade that, when bound to its cofactor Va (FVa) on a membrane surface, cleaves the zymogen prothrombin to active protease thrombin. FXa in turn is synthesized as the zymogen factor X (FX) that can be activated by either the intrinsic or extrinsic “tenase” complexes. Human FX is translated as a single polypeptide chain that is proteolytically processed into two disulfide-linked subunits: a 139 residue “light chain” and a 306 residue “heavy chain” (see, e.g., FIG. 4). The light chain contains two EGF-2 homology domains and a γ-carboxyglutamic acid (Gla) domain that is characteristic of vitamin K-dependent clotting factors and is primarily responsible for the membrane binding properties of FX/FXa. The heavy chain contains the serine protease domain as well as a 52-amino acid “activation peptide” at its amino terminus.

Because of the substantial homology between chymotrypsin-like serine proteases, a standard nomenclature has been developed based on the residue numbering of chymotrypsin that allows for more meaningful comparison of residues between different proteins. For FX, the amino-terminal light chain is non-homologous with chymotrypsin (since it contains the Gla domain and two EGF-2 domains not found in chymotrypsin) and is thus numbered sequentially from 1-139. Light chain residue numbers may be denoted by an L preceding the residue number (for example, ArgL139). Homology with chymotrypsin exists in the heavy chain which contains the protease domain and thus the heavy chain may be numbered in brackets according to the chymotrypsin numbering system (for example, Ser[195]).

Limited proteolysis by the intrinsic or extrinsic Xase complexes results in removal of the activation peptide, and subsequent conformational rearrangement, the “zymogen-to-protease transition,” of the protein results in the mature protease. Virtually all of the conformational change occurs in a distinct region of FXa known as the activation domain. Importantly, the zymogen-to-protease transition results in acquisition of three characteristic functional properties of FXa that are not present in the zymogen: 1) the ability to bind FVa, 2) the ability to bind prothrombin, and 3) a functional active site (see, e.g., Furie et al. (1976) J. Biol. Chem., 251:6807-6814; Robison et al. (1980) J. Biol. Chem., 255:2014-2021; Keyt et al. (1982) J. Biol. Chem., 257:8687-8695; Persson et al. (1991) J. Biol. Chem., 266:2458; Persson et al. (1993) J. Biol. Chem., 268:22531-22539; Dahlback et al. (1978) Biochem., 17:4938-4945). Upon removal of the activation peptide of FX, the newly exposed amino terminus of the heavy chain inserts into a hydrophobic pocket and forms a salt bridge between Ile[16] (the N-terminal residue) and Asp[194]. This leads to a series of conformational changes that yield the mature protease. The residues (H₂N-IVGG-; SEQ ID NO: 1) at the new N-terminus are highly conserved through evolution in FX/Xa, as is Asp[194]. In wild-type FXa, the protease conformation is in equilibrium with the zymogen conformation, with the equilibrium lying far towards the protease. Mutagenesis of Asp[194] in FXa can results in a catalytically inactive protein. Furthermore, through mutagenesis of the Ile[16] and Val[17] residues, it has been demonstrated that disruption of N-terminal insertion can shift the equilibrium between zymogen and protease towards the zymogen-like conformation. These “zymogen-like” FXa variants have a poorly formed active site and prothrombin binding site. However, binding of the cofactor FVa to these zymògen-like variants can thermodynamically rescue active protease conformation and function, indicating that cofactor binding is thermodynamically linked to the zymogen-to-protease transition. It has also been shown that strong ligands which bind to the activation domain of the zymogen, stabilize this region and at least partially mimic the changes seen in the zymogen to protease transition. Additionally, it has been shown that IVGG (SEQ ID NO: 1) peptides can at least partially activate trypsinogen (zymogen) in the absence of cleavage at position 16.

Wild-type (WT) FXa has a half-life in plasma of approximately 1 minute due to rapid inhibition by circulating plasma inhibitors. Serpins, irreversible serine protease inhibitors that include antithrombin III (ATIII), constitute a major class of these molecules. Tissue factor pathway inhibitor (TFPI) is another major plasma inhibitor of FXa. TFPI reversibly binds to and inhibits FXa, and forms a stable inactive quaternary complex upon further binding of tissue factor/FVIIa. Formation of this quaternary complex is irreversible. Both ATIII and TFPI interact with FXa at the substrate binding cleft and require a fully-formed active site. Furthermore, binding of small chromogenic substrates and these inhibitors to FXa is mutually exclusive. The N-terminal FXa zymogen-like variants described herein have been shown to be less susceptible to ATIII and TFPI inhibition and, as a result, have much longer half-lives.

The N-terminal zymogen-like variants of FXa are therapeutic procoagulants in the setting of excess bleeding such as with hemophilia and intracranial hemorrhage. Two key properties of these variants make them attractive as procoagulants. First, their longer half-lives make them more suitable pharmacologic agents than WT FXa. Second, they circulate predominantly in a zymogen-like conformation but are rescued at the site of vascular injury by binding to FVa. Thus, they are relatively inert while circulating free in plasma, but are functional proteases in the presence of FVa. Moreover, as demonstrated hereinbelow, FXa and FXa zymogen-like variants may also function as reversal agents for direct FXa inhibitors. Indeed, it is shown herein that FXa variants restore normal hemostasis in in vitro coagulation studies of plasma and whole blood anticoagulated with direct FXa inhibitors. Furthermore, since direct FXa inhibitors and TFPI/ATIII all bind in the substrate binding cleft of FXa, it is shown that direct FXa inhibitors prolong the half-life of FXa and variants thereof. It is also shown that FXa and FXa zymogen-like variants can safely and effectively reverse the effects of direct FXa inhibitors in vivo using murine hemostasis assays.

The instant invention encompasses FX molecules and variant FX molecules including FXa variants, FX variants, FX prepropeptide variants, and FX propeptide variants. For simplicity, the variants are generally described throughout the application in the context of FXa. However, the invention contemplates and encompasses FX, FX prepropeptide, and FX propeptide molecules, optionally having the same amino acid substitutions as the variant FXa.

The FXa and variants thereof of the instant invention can be from any mammalian species. In a particular embodiment, the FXa or variant thereof is human. GenBank Accession No. NP_000495 provides an example of the wild-type human FX preproprotein. FIG. 4A provides SEQ ID NO: 2, which is an example of the amino acid sequence of the human FX preproprotein. The FX prepropetide comprises a signal peptide from amino acids 1-23 and a propeptide sequence from amino acids 24-40. The cleavage of the propeptide yields a protein with a new N-terminus sequence of Ala-Asn-Ser. The FX prepropeptide is also cleaved into a mature two-chain form (light and heavy) by the excision at the tripeptide RKR to generate the Factor X zymogen. The two chains are linked via a disulfide bond. FIG. 4B provides SEQ ID NOs: 3 and 4, which are examples of the amino acid sequence of the human FX light and heavy chains, respectively. Factor X is activated by the cleavage of the 52 amino acid activation peptide to yield a new amino-terminal sequence of IVGG (SEQ ID NO: 1) for the wild-type FXa heavy chain. FIG. 4C provides SEQ ID NOs: 3 and 5, which are examples of the amino acid sequence of the human FXa light and heavy chains. Notably, the above proteolytic cleavage events may be imprecise, thereby leading to addition or loss of amino acids at the cleavage sites. For example, the mature FXa or variant thereof may be shorter than the predicted amino acid sequence due to imprecise proteolytic processing and maturation of the protein. FIG. 4D provides a nucleic acid sequence (SEQ ID NO: 6) which encodes human FX preproprotein. Nucleic acid molecules which encode FX and FXa can be readily determined from the provided amino acid and nucleotide sequences.

In a particular embodiment, the FX of the instant invention has at least 75%, 80%, 85%, 90%, 95%, 97%, 99%, or 100% homology (identity) with SEQ ID NO: 2, particularly at least 90%, 95%, 97%, or 99% homology. In a particular embodiment, the FX of the instant invention has at least 75%, 80%, 85%, 90%, 95%, 97%, 99%, or 100% homology with amino acids 24-488 of SEQ ID NO: 2, particularly at least 90%, 95%, 97%, or 99% homology. In a particular embodiment, the FX of the instant invention has at least 75%, 80%, 85%, 90%, 95%, 97%, 99%, or 100% homology with amino acids 41-488 of SEQ ID NO: 2, particularly at least 90%, 95%, 97%, or 99% homology. In a particular embodiment, the FX comprises a light and heavy chain, wherein the light chain has at least 75%, 80%, 85%, 90%, 95%, 97%, 99%, or 100% homology with SEQ ID NO: 3, particularly at least 90%, 95%, 97%, or 99% homology, and wherein the heavy chain has at least 75%, 80%, 85%, 90%, 95%, 97%, 99%, or 100% homology with SEQ ID NO: 4, particularly at least 90%, 95%, 97%, or 99% homology. In a particular embodiment, the FXa comprises a light and heavy chain, wherein the light chain has at least 75%, 80%, 85%, 90%, 95%, 97%, 99%, or 100% homology with SEQ ID NO: 3, particularly at least 90%, 95%, 97%, or 99% homology, and wherein the heavy chain has at least 75%, 80%, 85%, 90%, 95%, 97%, 99%, or 100% homology with SEQ ID NO: 5, particularly at least 90%, 95%, 97%, or 99% homology. The variants may vary by insertion, deletion, and/or substitution of one or more amino acids.

The variants of the instant invention may also be posttranslationally modified (γ-carboxylation). The variants may be posttranslationally modified in a cell or in vitro.

In a particular embodiment, the variants of the instant invention have an increased half-life in plasma (e.g., hemophilia plasma). Further, the variants of the invention in the absence of FVa may be refractory to all active site function and may be poor activators. However, the variants may exhibit activity in the presence of FVa.

The FXa variants of the instant invention may comprise at least one substitution at position 16, 17, 18, 19, and/or 194 (by chymotrypsin numbering; e.g., positions 235-239 and 418 in FIG. 4A (SEQ ID NO: 2)), particularly at position 16 or 17. Examples of FXa variants are also described in PCT/US2006/060927 and PCT/US2012/058279, both of which are incorporated by reference herein. In a particular embodiment, the isoleucine at position 16 is substituted with leucine, phenylalanine, aspartic acid, glycine, methionine, threonine, or serine. In a particular embodiment, the isoleucine at position 16 is substituted with leucine. In a particular embodiment, the isoleucine at position 16 is substituted with threonine. In a particular embodiment, the valine at position 17 is substituted with leucine, alanine, glycine, methionine, threonine, or serine. In a particular embodiment, the valine at position 17 is substituted with alanine. In a particular embodiment, the valine at position 17 is substituted with the hydroxyl amino acid threonine or serine. In a particular embodiment, the valine at position 17 is substituted with threonine. In a particular embodiment, the Asp at position 194 may be replaced with an asparagine or glutamic acid. The variants of the instant invention may comprise one or more of the above substitutions.

It will be appreciated by persons skilled in the art that variants (e.g., natural allelic variants) of Factor X/Xa sequences exist, for example, in the human population. Accordingly, it is within the scope of the present invention to encompass such variants, with respect to the Factor X/Xa amino acid and nucleotide sequences disclosed herein. Accordingly, the term “natural allelic variants” is used herein to refer to various specific nucleotide sequences of the invention and variants thereof that would occur in a human population. The usage of different wobble codons and genetic polymorphisms which give rise to conservative or neutral amino acid substitutions in the encoded protein are examples of such variants. Such variants would not demonstrate substantially altered Factor X/Xa activity or protein levels.

In a particular embodiment, the FXa variant is “zymogen-like” and has poor active site function and low reactivity towards the physiological inhibitors antithrombin III (ATIII) and tissue factor pathway inhibitor (TFPI). The biological activity of the variant may be at least mostly or fully rescued when associated with the cofactor FVa to form prothrombinase. For examples, FXaI16L can restore thrombin generation in hemophilic plasma and has a prolonged half-life (˜120 min vs. 1 min for wt-FXa; Toso, et al. (2008) JBC 283:18627-35; Bunce et al. (2011) Blood, 117:290-298). Furthermore, in vivo experiments with hemophilia B (HB) mice show that zymogen-like FXaI16L appears safe and provides adequate hemostasis in multiple injury models (Ivanciu and Camire, ASH Abstract, 2008; ISTH Abstract, 2009).

Nucleic acid molecules (e.g., cDNA, genomic DNA, or RNA) encoding the above proteins are also encompassed by the instant invention. Nucleic acid molecules encoding the proteins may be prepared by any method known in the art. The nucleic acid molecules may be maintained in any convenient vector, such as an expression vector or cloning vector. For example, clones may be maintained in a plasmid cloning/expression vector which may be propagated in a suitable host cell (e.g., E. coli or mammalian cells). In cases where post-translational modification affects function, it is preferable to express the molecule in mammalian cells.

In one embodiment, the nucleic acids encoding the FXa or variants thereof of the instant invention may be further modified via insertion of an intracellular proteolytic cleavage site (the instant invention also encompasses the resultant polypeptide both before and after cleavage). In order to express FXa variants in mammalian cells, a proteolytic cleavage site (e.g., an intracellular proteolytic cleavage site) can be inserted such that cleavage occurs between positions Arg15 and Ile16 in the FX (e.g., the cleavage site may be inserted between Arg15 and Ile16 or the entire 52 amino acid activation peptide can be replaced by the cleavage site). In a particular embodiment, the intracellular cleavage site is a PACE/furin-like enzyme cleavage site (e.g, Arg-X-(Arg/Lys)-Arg (SEQ ID NO: 7); Arg-Lys-Arg; or Arg-Lys-Arg-Arg-Lys-Arg (SEQ ID NO: 8)). The inclusion of the cleavage site at this location results in a processed FXa in which the heavy chain on the molecule begins at position 16. In other words, introduction of this cleavage site at this position will allow for the intracellular conversion of FX to FXa. These modifications allow for secretion of the “active” processed form of FX from a mammalian cell (e.g., CHO or Hela cells) that expresses the modified FX. Secretion of the cleaved factor obviates a need for proteolytic cleavage during blood clotting or following the isolation of the protein.

The proteins of the present invention may be prepared by any method known in the art, including isolation and purification from appropriate sources (e.g., transformed bacterial or animal cultured cells, or tissues which express variants). The proteins (e.g., those produced by gene expression in a recombinant prokaryotic or eukaryotic system) may be purified according to methods known in the art. In a particular embodiment, an expression/secretion system can be used, whereby the recombinant protein is expressed and thereafter secreted from the host cell, to be easily purified from the surrounding medium. The proteins may also be purified using affinity separation, such as by immunological interaction with antibodies that bind specifically to the recombinant protein or the use of a tag (e.g., 6-8 histidine residues, FLAG epitope, GST or the hemagglutinin epitope) on the protein, if present. Proteins, prepared by the aforementioned methods, may be analyzed according to standard procedures. For example, such proteins may be subjected to amino acid sequence analysis, according to known methods. The protein may also be subjected to the conventional quality controls and fashioned into a therapeutic form of presentation. In particular, during the recombinant manufacture, the purified preparation may be tested for the absence of cellular nucleic acids as well as nucleic acids that are derived from the expression vector

In a particular embodiment, the FX of the instant invention may be combined with Factor XIa or a derivative thereof, which is able to activate the FX variant into FXa. The FXa variant can be made available in the form of a combination preparation comprising a container that holds Factor XIa which may be in solution of immobilized on a matrix, potentially in the form of a miniature column or a syringe complemented with a protease, and a container containing the pharmaceutical preparation with the FX. To activate the FX, the factor X-containing solution, for example, can be pressed over the immobilized protease. During storage of the preparation, the FX-containing solution may be spatially separated from the protease. The preparation according to the present invention can be stored in the same container as the protease, but the components are spatially separated by an impermeable partition which can be easily removed before administration of the preparation. The solutions can also be stored in separate containers and be brought into contact with each other only shortly prior to administration. The FX can be activated into Factor Xa shortly before immediate use, e.g., prior to the administration to the patient. The activation can be carried out by bringing a factor X variant into contact with an immobilized protease or by mixing solutions containing a protease and the FX. Thus, it is possible to separately maintain the two components in solution and to mix them by means of a suitable infusion device in which the components come into contact with each other as they pass through the device and thereby to cause an activation into Factor Xa or into the Factor Xa variant. The patient thus receives a mixture of Factor Xa and, in addition, a serine protease which is responsible for the activation. In this context, it is especially important to pay close attention to the dosage since the additional administration of a serine protease also activates endogenous FX, which may shorten the coagulation time.

In a particular embodiment, the compositions of the instant invention comprise between about 10-5000 μg/kg, about 10-1000 m/kg, about 10-500 μg/kg, about 10-250 μg/kg, about 10-75 μg/kg, or about 40 μg/kg of the FXa or variant thereof. The amounts may be administered intravenously on an “as needed” basis or may be delivered on a schedule (e.g., at least one a day). Patients may be treated immediately upon presentation at the clinic with a bleed or prior to the delivery of cut/wound causing a bleed. Alternatively, patients may receive a bolus infusion every one to three hours, or if sufficient improvement is observed, a once daily infusion of the variant described herein.

As used herein, a “direct Factor Xa inhibitor” refers to a compound which selectively binds and inhibits Factor Xa directly. In a particular embodiment, the direct Factor Xa inhibitor possesses no inhibitory activity towards thrombin. Examples of direct Factor Xa inhibitors include, but are not limited to, antistasin, tick anticoagulant peptide, apixaban, betrixaban, darexaban, edoxaban, otamixaban, rivaroxaban, DX-9065a, YM-60828, RPR-120844, BX-807834, YM-150, PD-348292, razaxaban, BAY 59-7939, TAK-442, eribaxaban, LY517717, GSK913893, and salts, analogs, or derivatives thereof. Factor Xa inhibitors are also provided in U.S. Pat. Nos. 6,369,080; 6,262,047; and 6,133,256, incorporated herein by reference. In a particular embodiment, the direct Factor Xa inhibitor is selected from the group consisting of apixaban, betrixaban, darexaban, edoxaban, otamixaban, and rivaroxaban.

In accordance with the instant invention, methods of inhibiting, treating, and/or preventing a hemostasis related disease or disorder are provided. In a particular embodiment, the methods of the instant invention promote clot formation (procoagulation). In a particular embodiment, the methods of the instant invention neutralize and/or reverse the activity of an anticoagulant, particularly direct FXa inhibitors. In a particular embodiment, the FXa or variant thereof to inhibitor ratio is about 1:1 or 1:2 to about 1:10,000, particularly about 1:10 to about 1:1000, about 1:10 to about 1:500, or about 1:10 to about 1:100.

The instant invention encompasses methods of inhibiting, treating, and/or preventing a hemostasis related disease or disorder. Examples of hemostasis related diseases or disorders include, without limitation: bleeding disorders such as, without limitation, hemophilia, hemophilia A, hemophilia B, hemophilia A and B patients with inhibitory antibodies, deficiencies in at least one coagulation factor (e.g., Factors VII, IX, X, XI, V, XII, II, and/or von Willebrand factor), combined FV/FVIII deficiency, vitamin K epoxide reductase C1 deficiency, gamma-carboxylase deficiency; bleeding such as bleeding associated with, for example, trauma, injury, thrombosis, thrombocytopenia, stroke, coagulopathy (hypocoagulability), and/or disseminated intravascular coagulation (DIC); over-anticoagulation such as over-anti-coagulation with heparin, low molecular weight heparin, pentasaccharide, warfarin, and/or small molecule antithrombotics (e.g., FXa inhibitors); and platelet disorders such as, without limitation, Bernard Soulier syndrome, Glanzman thromblastemia, and storage pool deficiency. In a particular embodiment, the method comprises administering to a subject in need thereof a therapeutically effective amount of: 1) at least one FXa or variant thereof and 2) at least one direct FXa inhibitor. The compounds may be administered simultaneously and/or sequentially. The FXa or variant thereof and direct FXa inhibitor may be contained in the same composition (e.g., with at least one pharmaceutically acceptable carrier) or be present in separate compositions (e.g., with the same or different pharmaceutically acceptable carrier). The composition(s) may comprise at least one carrier, particularly at least one pharmaceutically acceptable carrier. When the compositions are administered separately, the compositions may be administered simultaneously and/or sequentially. For example, the FXa or variant thereof may be administered first and then the direct FXa inhibitor; the direct FXa inhibitor may be administered first and then the FXa or variant thereof; or multiple administrations of each component may be used in any order. The methods of the instant invention may further comprise administering other therapies which are beneficial to the treatment of the particular hemostasis related disease or disorder. For example, the FXa or variant thereof may be administered with at least one other agent known to modulate hemostasis (e.g., Factor V, Factor Va, or derivatives thereof). In a particular embodiment, the FXa or variant thereof to inhibitor ratio is about 1:1 or 1:2 to about 1:10,000, particularly about 1:10 to about 1:1000, about 1:10 to about 1:500, or about 1:10 to about 1:100.

In a particular embodiment, the hemostasis related disease or disorder is over anti-coagulation with a direct Factor Xa inhibitor. When the disease or disorder excess anti-coagulation due to the use/presence of Factor Xa inhibitors (e.g., the excess use/presence of a Factor Xa inhibitor leading to bleeding), the anti-coagulation caused by the Factor Xa inhibitor (e.g., direct FXa inhibitor) can be inhibited, treated, and/or prevented by delivering at least one FXa or variant thereof to the blood (e.g., by administering at least one FXa or variant thereof to the subject). In a particular embodiment, at least one FXa variant is administered to the subject.

The instant invention also encompasses methods of increasing the coagulation of blood. In a particular embodiment, the method comprises contacting the blood with 1) at least one FXa or variant thereof and 2) at least one direct FXa inhibitor. The method may be performed in vitro or in vivo. The compounds may be delivered to the blood simultaneously and/or sequentially. The FXa or variant thereof and direct FXa inhibitor may be contained in the same composition (e.g., with at least one carrier) or be present in separate compositions (e.g., with the same or different carrier). The composition(s) may comprise at least one carrier (e.g., at least one pharmaceutically acceptable carrier). When the compositions are delivered separately, the compositions may be administered simultaneously and/or sequentially. For example, the FXa or variant thereof may be delivered first and then the direct FXa inhibitor; the direct FXa inhibitor may be delivered first and then the FXa or variant thereof; or multiple deliveries of each component may be used in any order. The methods of the instant invention may further comprise delivering at least one other agent known to modulate hemostasis (e.g., Factor V, Factor Va, or derivatives thereof).

In accordance with another aspect of the instant invention, compositions comprising at least one FXa or variant thereof and at least one direct FXa inhibitor are provided. The compositions may be used for the treatment/inhibition/prevention of a hemostasis related disease or disorder or may be used to promote coagulation of blood. In one embodiment, the composition further comprises at least one pharmaceutically acceptable carrier. In a particular embodiment, the instant invention encompasses a kit comprising at least two compositions: wherein one composition comprises at least one FXa or variant thereof and, optionally, at least one pharmaceutically acceptable carrier; and the second composition comprises at least one direct FXa inhibitor and, optionally, at least one pharmaceutically acceptable carrier. The kit may further comprise at least one other agent known to modulate hemostasis (e.g., Factor V, Factor Va, or derivatives thereof), optionally present in a composition with a pharmaceutically acceptable carrier.

The compositions of the instant invention may comprise a physiologically acceptable matrix. The pharmaceutical compositions of the present invention can be administered to the blood by any suitable route, for example, by infusion, injection or other modes of administration such as controlled release devices. In a particular embodiment, the composition is delivered by intravenous injection. The compositions of the instant invention may be directly administered or applied to the site of bleeding (e.g., by injection). In general, pharmaceutical compositions and carriers of the present invention comprise, among other things, pharmaceutically acceptable buffers, diluents, liquids (such as water, saline, glycerol, sugars and ethanol), preservatives, stabilizing agents, solubilizers, emulsifiers, wetting agents, pH buffering substances adjuvants and/or carriers. Such compositions can include diluents of various buffer content (e.g., saline, Tris HCl, acetate, phosphate), pH and ionic strength; and additives such as detergents and solubilizing agents (e.g., Tween 80, Polysorbate 80), anti oxidants (e.g., ascorbic acid, sodium metabisulfite), preservatives (e.g., Thimersol, benzyl alcohol) and bulking substances (e.g., lactose, mannitol). For example, the preparation can be formulated with a buffer containing salts, such as NaCl, CaCl₂, and amino acids, such as glycine and/or lysine, and in a pH range from 6 to 8. The pharmaceutical compositions may be formulated in aqueous solutions (e.g., physiologically compatible buffers). Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions. The compositions of the invention may also be incorporated into particulate preparations of polymeric compounds such as polylactic acid, polyglycolic acid, etc., or into liposomes or micelles, or mixed with phospholipids or micelles to increase stability. Such compositions may influence the physical state, stability, rate of in vivo release, and rate of in vivo clearance of components of a pharmaceutical composition of the present invention. Exemplary pharmaceutical compositions and carriers are provided, e.g., in “Remington's Pharmaceutical Sciences” by E. W. Martin (Mack Pub. Co., Easton, Pa.) and “Remington: The Science And Practice Of Pharmacy” by Alfonso R. Gennaro (Lippincott Williams & Wilkins) which are herein incorporated by reference. The pharmaceutical composition of the present invention can be prepared, for example, in liquid form, deep-frozen, or can be in dried powder form (e.g., lyophilized). In a particular embodiment, when the preparation is stored in lyophilized form, it may be dissolved into a visually clear solution using an appropriate reconstitution solution prior to administration.

The compositions described herein will generally be administered to a patient as a pharmaceutical preparation. The term “patient” or “subject”, as used herein, refers to human or animal subjects. The compositions of the instant invention may be employed therapeutically, under the guidance of a physician.

The compositions of the instant invention may be conveniently formulated for administration with any carrier, particularly any pharmaceutically acceptable carrier(s). Except insofar as any conventional carrier is incompatible with the agents to be administered, its use in the pharmaceutical composition is contemplated. For example, the active agents may be formulated with an acceptable medium such as sterile liquid, water, aqueous solutions, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the active agents in the chosen medium may be varied and the medium may be chosen based on the desired route of administration of the pharmaceutical preparation. Except insofar as any conventional media or agent is incompatible with the active agents to be administered, its use in the pharmaceutical preparation is contemplated.

The dose and dosage regimen of the compositions according to the invention that are suitable for administration to a particular patient may be determined by a physician considering the patient's age, sex, weight, general medical condition, and the specific condition for which the active agent is being administered and the severity thereof (e.g., the severity of the bleeding). The physician may also take into account the route of administration, the pharmaceutical carrier, and the particular agent's biological activity.

Selection of a suitable pharmaceutical preparation will also depend upon the mode of administration chosen. For example, the compositions of the invention may be administered by direct injection to a desired site. In this instance, a pharmaceutical preparation comprises the active agents of the instant invention dispersed in a medium that is compatible with the site of injection. The compositions of the instant invention may be administered by any method. For example, the compositions can be administered, without limitation, intravenously. Pharmaceutical preparations for injection are known in the art. If injection is selected as a method for administering the compositions, steps must be taken to ensure that sufficient amounts of the molecules reach their target cells to exert a biological effect.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of the composition may be determined by evaluating the toxicity of the molecules or cells in animal models. Various concentrations of active agents in pharmaceutical preparations may be administered to mice or other animal models, and the minimal and maximal dosages may be determined based on the beneficial results and side effects observed as a result of the treatment. Appropriate dosage unit may also be determined by assessing the efficacy of the treatment in combination with other standard drugs. The dosage units of the compositions of the instant invention may be determined individually or in combination with each treatment according to the effect detected.

While the administration of FXa protein or a variant thereof is described hereinabove, nucleic acids encoding the FXa (or FX) or variant thereof may be used. In a particular embodiment of the invention, a nucleic acid delivery vehicle (e.g., an expression vector) for modulating blood coagulation is provided wherein the nucleic acid delivery vehicle comprises a nucleic acid sequence coding for FXa or a variant thereof. Administration of FXa-encoding expression vectors to a patient results in the expression of FXa polypeptide or a variant thereof which serves to alter the coagulation cascade. In accordance with the present invention, a FXa or variant thereof encoding nucleic acid sequence may encode a variant polypeptide as described herein whose expression increases clot formation. As with the administration of the protein, expression vectors comprising FXa or variant nucleic acid sequences may be administered alone, or in combination with other molecules useful for modulating hemostasis. According to the present invention, the expression vectors or combination of therapeutic agents may be administered to the patient alone or in a pharmaceutically acceptable or biologically compatible composition.

In a particular embodiment of the invention, the expression vector comprising nucleic acid sequences encoding the FXa or variant is a viral vector. Viral vectors which may be used in the present invention include, but are not limited to, adenoviral vectors (with or without tissue specific promoters/enhancers), adeno-associated virus (AAV) vectors of multiple serotypes (e.g., AAV-2, AAV-5, AAV-7, and AAV-8) and hybrid AAV vectors, lentivirus vectors and pseudo-typed lentivirus vectors [e.g., Ebola virus, vesicular stomatitis virus (VSV), and feline immunodeficiency virus (FIV)], herpes simplex virus vectors, vaccinia virus vectors, and retroviral vectors.

In a particular embodiment of the present invention, methods are provided for the administration of a viral vector comprising nucleic acid sequences encoding a variant or a functional fragment thereof. Adenoviral vectors of utility in the methods of the present invention preferably include at least the essential parts of adenoviral vector DNA. As described herein, expression of a variant polypeptide following administration of such an adenoviral vector serves to modulate hemostasis, particularly to enhance the procoagulation activity of the protease. Recombinant adenoviral vectors have found broad utility for a variety of gene therapy applications. Their utility for such applications is due largely to the high efficiency of in vivo gene transfer achieved in a variety of organ contexts.

The vectors of the present invention may be incorporated into pharmaceutical compositions that may be delivered to a subject, so as to allow production of a biologically active protein (e.g., a variant polypeptide or functional fragment or derivative thereof). In a particular embodiment of the present invention, pharmaceutical compositions comprising sufficient genetic material to enable a recipient to produce a therapeutically effective amount of a variant polypeptide can influence hemostasis in the subject. Alternatively, as discussed above, an effective amount of the variant polypeptide may be directly infused into a patient in need thereof. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents (e.g., co-factors) which influence hemostasis.

### DEFINITIONS

Various terms relating to the biological molecules of the present invention are used hereinabove and also throughout the specification and claims.

The singular forms “a,” “an,” and “the” include plural referents unless the context clearly dictates otherwise.

The phrase “hemostasis related disorder” refers to bleeding disorders such as, without limitation, hemophilia A, hemophilia B, hemophilia A and B patients with inhibitory antibodies, deficiencies in at least one coagulation factor (e.g., Factors VII, IX, X, XI, V, XII, II, and/or von Willebrand factor), combined FV/FVIII deficiency, vitamin K epoxide reductase C1 deficiency, gamma-carboxylase deficiency; bleeding associated with trauma, injury, thrombosis, thrombocytopenia, stroke, coagulopathy (hypocoagulability), disseminated intravascular coagulation (DIC); over-anticoagulation associated with heparin, low molecular weight heparin, pentasaccharide, warfarin, small molecule antithrombotics (e.g., direct FXa inhibitors); and platelet disorders such as, Bernard Soulier syndrome, Glanzman thromblastemia, and storage pool deficiency.

With reference to nucleic acids of the invention, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the DNA of a prokaryote or eukaryote.

With respect to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

With respect to protein, the term “isolated protein” is sometimes used herein. This term may refer to a protein produced by expression of an isolated nucleic acid molecule of the invention. Alternatively, this term may refer to a protein which has been sufficiently separated from other proteins with which it would naturally be associated (e.g., so as to exist in “substantially pure” form).

The term “vector” refers to a carrier nucleic acid molecule (e.g., DNA) into which a nucleic acid sequence can be inserted for introduction into a host cell where it will be replicated. An “expression vector” is a specialized vector that contains a gene or nucleic acid sequence operably linked to the necessary regulatory regions needed for expression in a host cell.

The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “substantially pure” refers to a preparation comprising at least 50-60% by weight the compound of interest (e.g., nucleic acid, oligonucleotide, protein, etc.), particularly at least 75% by weight, or at least 90-99% or more by weight of the compound of interest. Purity may be measured by methods appropriate for the compound of interest (e.g. chromatographic methods, agarose or polyacrylamide gel electrophoresis, HPLC analysis, and the like).

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., bleeding, particularly uncontrolled bleeding (e.g., receive excess anti-coagulation drugs)) resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof. For example, “therapeutically effective amount” may refer to an amount sufficient to halt bleeding in a subject.

As used herein, the term “subject” refers to an animal, particularly a mammal, particularly a human.

The following example is provided to illustrate various embodiments of the present invention. The example is illustrative and is not intended to limit the invention in any way.

### Example

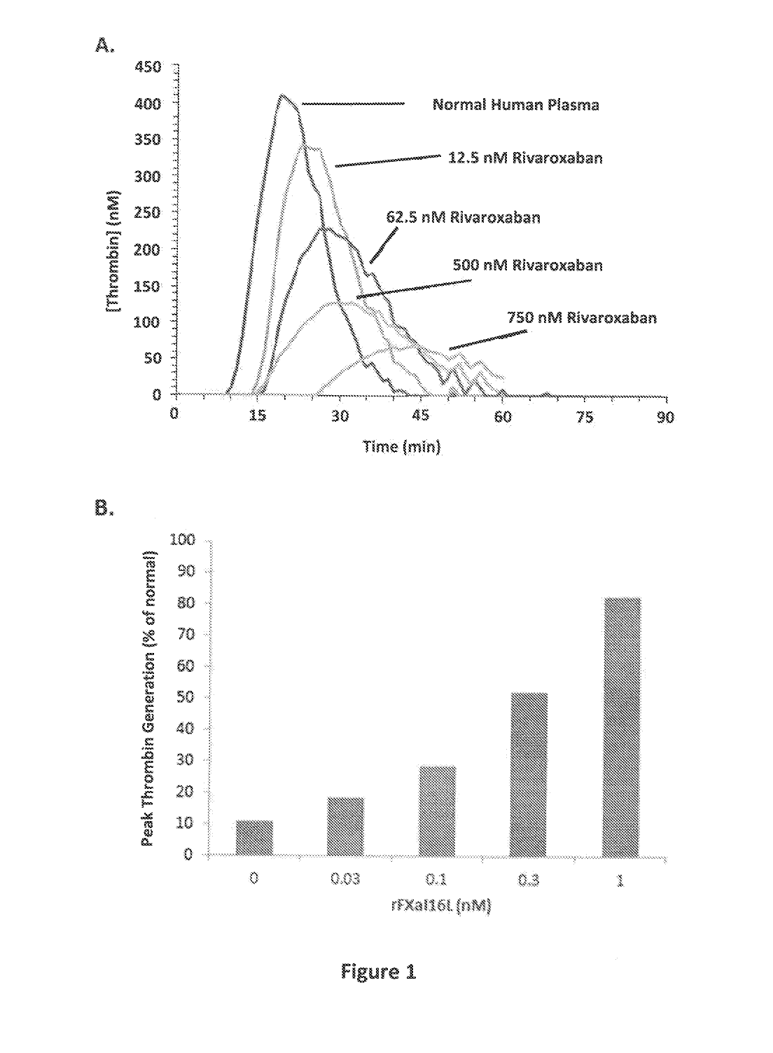

Rivaroxaban and apixaban at therapeutic plasma concentrations in normal human platelet poor plasma (PPP) profoundly decrease thrombin generation in thrombin generation assays (TGA) (FIG. 1A). TGAs are 96-well format assays that use a tissue-factor/phospholipid initiator and measure thrombin generation with a fluorogenic thrombin substrate. Upon titration of FXa^{I[16]L} at concentrations lower than 1% of the inhibitor concentration, thrombin generation is almost completely restored (FIG. 1B).

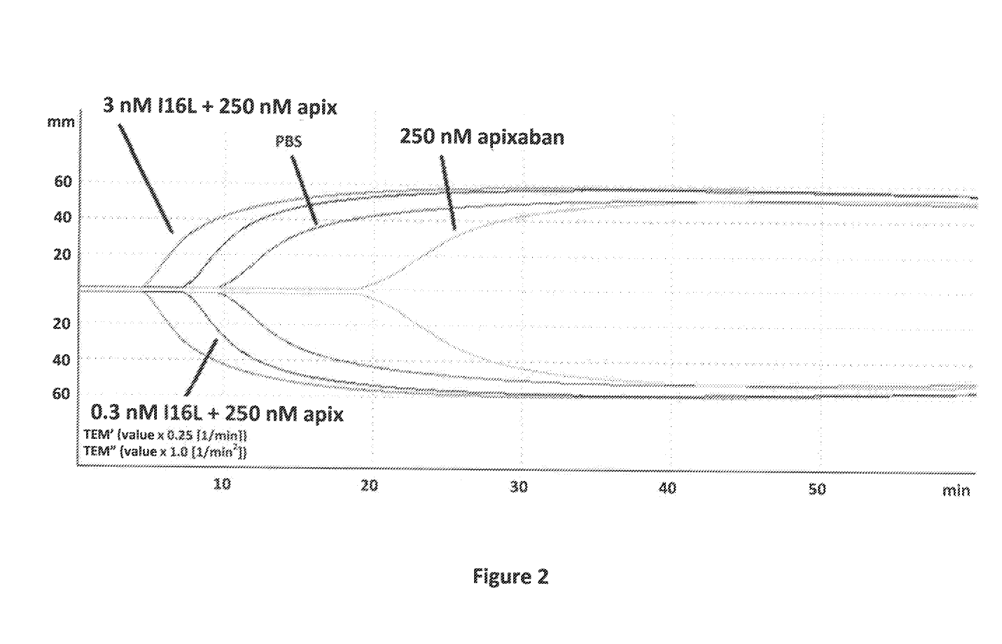

To determine if these results are recapitulated in whole blood, rotational thromboelastography (ROTEM) experiments were performed using apixaban and FXa^{I[16]L}. In ROTEM, a rotating pin is submerged in a cup containing whole blood. A coagulation initiator is added, and as the blood clots, rotation of the pin is restricted, which is detected optically by the instrument. In these experiments, 250 nM apixaban was added to citrated whole blood along with PBS or increasing concentrations of FXa^{I[16]L}. As shown in FIG. 2, apixaban prolonged the clot time compared to untreated control blood, and addition of FXa^{I[16]L} completely restored normal clot times.

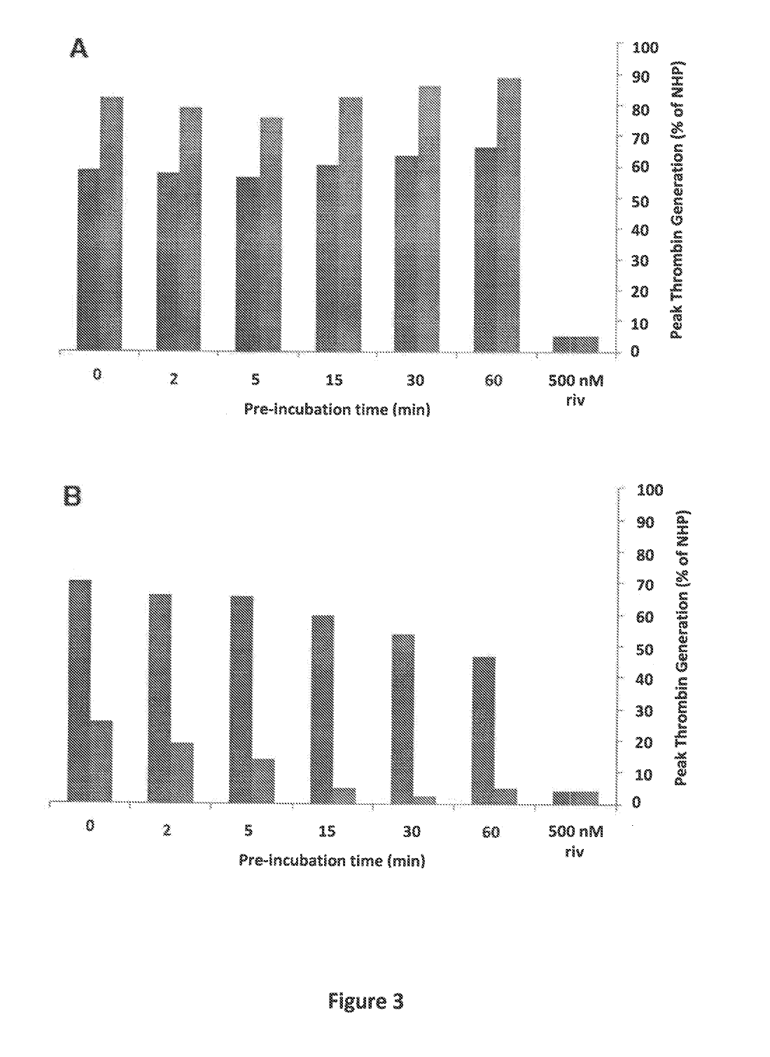

To determine if FXa half-life is prolonged in the presence of direct FXa inhibitors, half-life studies were performed by pre-incubating FXa^{I[16]L} or WT FXa in plasma in the presence of 500 nM rivaroxaban prior to initiating TGA reactions. Although the exact half-lives from this experiment could not be determined due to the limited 1 hour time course, FIG. 3 clearly demonstrates that the half-lives of both FXa^{I[16]L} and WT FXa are longer than 1 hour, substantially longer than the previously determined half-lives (e.g., >30 minutes in vitro for FXa^{I[16]L} and 1-2 minutes for WT FXa).

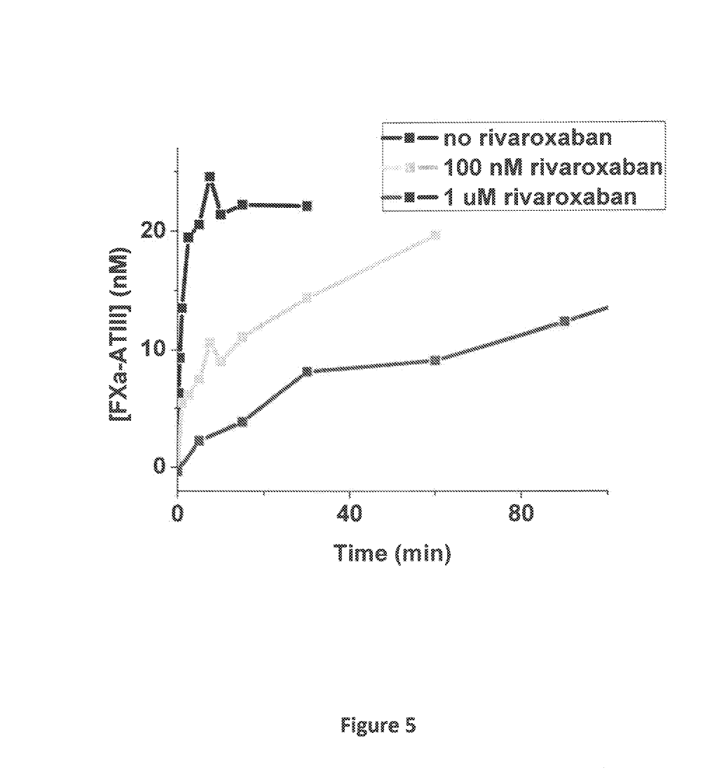

To further demonstrate that rivaroxaban prevents inhibition of FXa, FXa-antithrombin III (FXa-ATIII) levels were measured in plasma. As shown in FIG. 5, within 5 minutes, nearly all of the 25 nM WT FXa added to FX-deficient plasma became incorporated in an irreversible FXa-ATIII inhibitory complex. Rivaroxaban dose-dependently inhibited this process, such that, in the presence of 1 μM rivaroxaban, only about half of the added FXa was inactivated by ATIII after 90 minutes. Thus, FIG. 5 clearly shows that rivaroxaban inhibits FXa-ATIII complex formation.

As stated hereinabove, binding to FVa rescues the activity of the zymogen-like FXa variants and, as a result, they are highly effective procoagulants in vivo in the setting of hemophilia. Accordingly, these variants can also be effective procoagulants to overcome the effects of direct FXa inhibitors. Furthermore, since direct FXa inhibitors bind the FXa active site, the variants can compete with ATIII and TFPI for FXa binding and prolong their half-lives. Rivaroxaban dose-dependently inhibited thrombin generation in thrombin generation assays (TGA) when added to normal human plasma. Specifically, 500 nM rivaroxaban, the expected therapeutic steady-state plasma concentration, decreased peak thrombin generation to ˜10% of normal, and addition of 3 nM of the FXa zymogen-like variant FXa^{I16L} restored peak thrombin generation to 105% of normal. Higher concentrations of rivaroxaban (2.5 μM) completely abrogated thrombin generation in this assay, but 10 nM FXa^{I16L} restored thrombin generation to 72% of normal under these conditions. These data were compared to results obtained with other proposed reversal strategies. Gla-domainless, catalytically inactive FXa (GD-FXa^{S195A}), which has been shown to reverse the effects of rivaroxaban by scavenging the inhibitor, restored thrombin generation in the presence of 500 nM rivaroxaban, but required high concentrations (1 μM; >300-fold greater than FXaI16L) to be effective. In addition, activated prothrombin complex concentrates (FEIBA), which have been shown to have some ex vivo efficacy, did not restore thrombin generation under the present assay conditions.

In tail-clip hemostasis experiments in mice, rivaroxaban dose-dependently increased blood loss, with 50 mg/kg rivaroxaban resulting in 217% of normal blood loss. Addition of FXaI16L (200 mg/kg) reduced rivaroxaban-induced blood loss to 141% of normal. To examine the effect of rivaroxaban on the half-life of FXa, FXa^{I16L} or wt-FXa was pre-incubated with or without rivaroxaban in normal human plasma and then performed TGA experiments after various incubation times. When wt-FXa or FXa^{I16L} were pre-incubated in plasma in the absence of rivaroxaban, their half-lives were 4.6 minutes and 1.37 hours, respectively. Remarkably, when wt-FXa or FXaI16L were incubated in plasma in the presence of 500 nM rivaroxaban, their respective half-lives were prolonged to 9.4 hours (123-fold increase) and 18.1 hours (13.2-fold increase). These results indicate that a zymogen-like variant of FXa, FXa^{I16L} can reverse the effects of rivaroxaban in vitro and in vivo. Furthermore, FXa^{I16L} is a bypassing agent that only requires catalytic amounts of protein, in contrast to scavengers or “true” antidotes like GD-FXa^{S195A} which require stoichiometric concentrations for efficacy. This indicates that much lower quantities of FXa^{I16L} may be effective in vivo. It was also demonstrated that rivaroxaban dramatically prolongs the half-life of FXa in plasma, likely by competing with ATIII and TFPI for FXa binding. This demonstrates a long half-life reversal strategy for direct FXa inhibitors.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for treating a hemostasis related disorder in a subject in need thereof comprising simultaneously administering a therapeutically effective amount of Factor Xa or a variant thereof and a direct FXa inhibitor.

2. The method of claim 1, wherein said hemostasis related disorder is selected from the group consisting of hemophilia A, hemophilia B, hemophilia A and B associated with inhibitory antibodies, coagulation factor deficiency, vitamin K epoxide reductase C1 deficiency, gamma-carboxylase deficiency, bleeding associated with trauma or injury, thrombosis, thrombocytopenia, stroke, coagulopathy, disseminated intravascular coagulation (DIC), over-anticoagulation treatment disorders, Bernard Soulier syndrome, Glanzman thromblastemia, and storage pool deficiency.

3. The method of claim 2, wherein said coagulation factor deficiency is a deficiency of at least one coagulation factor selected from the group consisting of factor VII, factor IX, factor X, factor XI, factor V, factor XII, factor II, and von Willebrand factor.

4. The method of claim 2, wherein said over-anticoagulation treatment disorder results from the prior administration of at least one anticoagulant selected from the group consisting of heparin, low molecular weight heparin, pentasaccharide, warfarin, small molecule antithrombotics, and FXa inhibitors.

5. The method of claim 1, wherein said direct FXa inhibitor is selected from the group consisting of apixaban, betrixaban, darexaban, edoxaban, otamixaban, and rivaroxaban.

6. The method of claim 1, wherein said Factor Xa or a variant thereof comprises a light and heavy chain, wherein the light chain has at least 90% homology with SEQ ID NO: 3, and wherein the heavy chain has at least 90% homology with SEQ ID NO: 5.

7. The method of claim 1, wherein said Factor Xa or variant thereof comprises a Leu at position 16 in chymotrypsin numbering system.

8-11. (canceled)

12. A composition comprising at least one Factor Xa or variant thereof and at least one direct FXa inhibitor.

13. The composition of claim 12 further comprising at least one pharmaceutically acceptable carrier.

14. The composition of claim 12, wherein said direct FXa inhibitor is selected from the group consisting of apixaban, betrixaban, darexaban, edoxaban, otamixaban, and rivaroxaban.

15. The composition of claim 12, wherein said Factor Xa or a variant thereof comprises a light and heavy chain, wherein the light chain has at least 90% homology with SEQ ID NO: 3, and wherein the heavy chain has at least 90% homology with SEQ ID NO: 5.

16. The composition of claim 12, wherein said Factor Xa or variant thereof comprises a Leu at position 16 in chymotrypsin numbering system.


SEQUENCE LISTING (8 sequences)

SEQ ID NO: 1 - protein, 4 aa, Artificial Sequence
  N-terminus
IVGG

SEQ ID NO: 2 - protein, 488 aa, Homo sapiens
MGRPLHLVLLSASLAGLLLLGESLFIRREQANNILARVRRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 3 - protein, 139 aa, Homo sapiens
ANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLER

SEQ ID NO: 4 - protein, 306 aa, Homo sapiens
SVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 5 - protein, 254 aa, Homo sapiens [sequence omitted]

SEQ ID NO: 6 - DNA, 1467 bp, Homo sapiens
atggggcgcccactgcacctcgtcctgctcagtgcctccctggctggcctcctgctgctcggggaaagtctgttcatccgcagggagcaggccaacaacatcctggcgagggtcaggagggccaattcctttcttgaagagatgaagaaaggacacctcgaaagagagtgcatggaagagacctgctcatacgaagaggcccgcgaggtctttgaggacagcgacaagacgaatgaattctggaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggtcagtggcccaggccaccagcagcagcggggaggcccctgacagcatcacatggaagccatatgatgcagccgacctggaccccaccgagaaccccttcgacctgcttgacttcaaccagacgcagcctgagaggggcgacaacaacctcacgcgtatcgtgggaggccaggaatgcaaggacggggagtgtccctggcaggccctgctcatcaatgaggaaaacgagggtttctgtggtggaactattctgagcgagttctacatcctaacggcagcccactgtctctaccaagccaagagattcaaggtgagggtaggtgaccggaacacggagcaggaggagggcggtgaggcggtgcacgaggtggaggtggtcatcaagcacaaccggttcacaaaggagacctatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagctggggagagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaagtga

SEQ ID NO: 7 - protein, 4 aa, Artificial Sequence
  PACE/furin cleavage site
RXXR

SEQ ID NO: 8 - protein, 6 aa, Artificial Sequence
  PACE/furin cleavage site
RKRRKR